(19) United States
(12) Patent Application Publication
(10) Pub. No.: US 2013/0252257 A1
(43) Pub. Date: Sep. 26, 2013

(54) QUANTIFICATION OF CIRCULATING STEM AND PROGENITOR CELLS AS A MEANS OF ASSESSING EFFICACY OF NATUROPATHIC INTERVENTIONS

(71) Applicants: Amit Patel, Salt Lake City, UT (US); Annette M. Marleau, San Diego, CA (US)

(72) Inventors: Amit Patel, Salt Lake City, UT (US); Annette M. Marleau, San Diego, CA (US)

(73) Assignee: Creative Medical Health, Inc., Phoenix, AZ (US)

(21) Appl. No.: 13/621,702

(22) Filed: Sep. 17, 2012

Related U.S. Application Data
(60) Provisional application No. 61/535,752, filed on Sep. 16, 2011.

(51) Int. Cl. G01N 33/569

(52) U.S. Cl. CPC G01N 33/56966
USPC 435/7.21; 435/39

(57) ABSTRACT

Disclosed are tests, correlations and “theranostic” interventions aimed at optimizing general health through quantification of cells with regenerative potential in circulation. Some embodiments include use of circulating endothelial progenitor cell (EPC) testing as a means of quantifying general health status in a patient. The combination of EPC testing together with a naturopathic intervention, wherein the selection and dosage of said naturopathic intervention is tailored based on said quantitative test is provided. In other embodiments, regenerative cells are selected from the group comprising of very small embryonic like cells (VSEL), CD34 cells with hematopoietic potential, and circulating mesenchymal stem cells (MSC). Naturopathic interventions include recommendations of lifestyle modification, dietary supplements, intravenous vitamins, detoxification, acupuncture, and guided imagery.

### CROSS-REFERENCE TO RELATED APPLICATIONS

This application claims priority to co-pending U.S. Provisional Application Ser. No. 61/535,752, filed Sep. 16, 2011, which is expressly incorporated herein by reference in its entirety.

### FIELD OF THE INVENTION

The present technology pertains to the area of proactively identifying risk factors for disease, and proactively intervening. More specifically, the present technology relates to quantitative assessment of general health status, through the use of quantification of circulating regenerative cells. More specifically, the present technology provides means of quantifying susceptibility to disease and intervening either to prevent said disease, or to treat said disease state. In some embodiments the present technology relates to the field of theragnostics, that is, linking a diagnosis methodology with treatment of a disease and gauging the efficacy of the intervention at a cellular level before clinical manifestations are observed.

### BACKGROUND

The field of naturopathic medicine is historically based on clinical practice and observations. While numerous studies have demonstrated clinical effects of various naturopathic interventions, the mechanisms of these effects are often described as “cleansing the body,” “readjusting energy fields” or “detoxification.” Unfortunately, very few biomarkers exist to determine efficacy of a naturopathic intervention before clinical effect is observed. The present technology seeks to address this issue.

### SUMMARY OF THE INVENTION

The present technology provides methods, materials and compositions of matter of assessing general health, and by this means, efficacy of naturopathic and allopathic interventions. Without being limited thereto, emphasis is placed on naturopathic interventions due to the lack of quantitative outputs of efficacy before clinical responses are observed.

Accordingly, in some embodiments presented herein, methods are provided of assessing the general health of a mammal, the methods can include for example, one or more of: a) obtaining a blood sample; b) labeling said blood sample with an agent capable of selectively binding a cell with regenerative potential; c) quantifying a number of cells with regenerative potential; and d) relating said number of cells with regenerative potential with various aspects of general health.

Also presented herein are methods of assessing general health of a mammal, which methods can include, for example correlating levels of a regenerative cell in circulation together with levels of an inflammatory marker. In some embodiments, the levels of a regenerative cell in circulation are determined by: obtaining a blood sample; labeling said sample with an agent capable of selectively binding a cell with regenerative potential; quantifying said number of cells with said regenerative potential; and relating number of said cells with said regenerative potential with various aspects of general health.

Thus, in some aspects of the above-described embodiments, the agent capable of selectively binding a cell with regenerative potential can be or can include, for example, one or more of: a protein; a peptide; a nucleic acid; and a small molecule. In certain aspects, the agent capable of selectively binding a cell with regenerative potential is an antibody. In certain aspects, the agent capable of selectively binding a cell with regenerative potential is further may be conjugated to a label enabling detection, for example. In one aspect nucleic acids are used as a means of detection of cells with regenerative potential. In one specific aspect detection of mRNA is performed for transcripts encoding genes associated with regenerative cells. In one specific aspect detection of mRNA encoding for CD133, CD34 and VEGF receptors is performed. Means of performing this are commonly known in the art, with one example including RT-PCR.

In certain embodiments, the cell with regenerative potential can be, for example a circulating cell such as one or more of mesenchymal stem cells (MSC); endothelial progenitor cells (EPC); very small embryonic like cells (VSEL); and hematopoietic stem cells. In certain embodiments, the cell with regenerative potential can be, for example a circulating endothelial progenitor cell.

In some aspects, the mesenchymal stem cells maybe detected by an agent capable of binding a molecule, for example, one or more of: CD5; CD9; CD73; CD90; and CD105. In some aspects, the mesenchymal stem cells may be detected by the absence of expression of a marker, for example, one or more of: CD14; CD34; CD45; and HLA II.

In some aspects, the endothelial progenitor cells can be detected by an agent capable of binding a molecule, for example, one or more of: CD34; CD 133; KDR-1; and CD166.

In some aspects, the very small embryonic like cells can be detected based on a size of less than 7 microns, and if desired, also together with expression of a molecule such as, for example, one or more of: wnt-5; CD34; CD133; Oct-4; Nanog; and SSEA-1.

In some aspects, the hematopoietic stem cells may be detected based on expression of a molecule, for example, one or more of: CD34; CD133; c-kit; and jagged.

In some aspects, the hematopoietic stem cells can express less than 5% of markers, such as one or more of: CD14; CD38; and CD45, for example.

In some aspects, the mesenchymal stem cell can be capable of forming mesenchymal stem cells when cultured in liquid culture. Also, the mesenchymal stem cells can have the ability to differentiate into one or more of osteocytic, chondrocytic, and adipocytic lineages, for example.

In some aspects, the endothelial progenitor cell can be capable of forming endothelial cells when cultured in liquid culture. Said endothelial cells can express the ability to uptake acetylated LDL.

In some aspects, the very small embryonic like cell can be capable of forming multiple tissues in liquid culture. That capability can in addition to the capability of forming one or more of adipose, chondrogenic, and osteogenic tissues.

In some aspects, the hematopoietic stem cell can be capable of forming hematopoietic tissues in liquid culture. Said hematopoietic tissues may include, for example, one or more of granulocytes and monocytes.

In some aspects, assessment of circulating regenerative cells can be performed by one or more of: flow cytometry, enzyme linked immunosorbent assay (ELISA), Western Blot, reverse transcriptase polymerase chain reaction, can real-time RT-PCR, and the like.

In some aspects, general health can include for example, freedom from cardiovascular, neurological, ophthalmological, renal, hepatic, neurodegenerative, dermal and fibrotic disease. In some aspects, general health may include reduction of age-associated biological changes.

In embodiments where an inflammatory marker is correlated, the inflammatory marker can be a circulating marker, for example, one or more of: TNF-alpha, IL-1, IL-6, IL-8, IL-17, IL-21, IL-22, IL-27, IFN-gamma, neopterin, kynurenine, homocysteine, C-reactive protein, circulating complement degradation compounds, fibrinogen, D-Dimers, and HMGB-1.

In certain aspects, the inflammatory marker can be a specific property of circulating cells, said property associated with inflammation. In certain aspects, the property associated with inflammation can be, for example, one or more of: erythrocyte sedimentation, monocyte production of TNF-alpha, monocyte production of TNF-alpha after LPS stimulation, expression of TLR-2 on monocytes, and expression of TLR-4 on monocytes.

In one aspect assessment of regenerative cells is performed in circulation through the use of ELISA, Western Blot, Northern Blot, RT-PCR or real-time RT-PCR.

In one aspect of the invention, general health comprises susceptibility to and/or freedom from one or more of the disease conditions selected from: cardiovascular, neurological, ophthalmological, renal, hepatic, neurodegenerative, dermal and fibrotic disease. In one aspect, comparing said number of regenerative cells to a baseline level determined from said mammal is performed to evaluate general health.

In one aspect of the invention, a method of assessing general health of a mammal is disclosed comprising correlating levels of a regenerative cell in circulation together with levels of an inflammatory marker, wherein said inflammatory marker is a circulating marker selected from the group comprising of: TNF-alpha; IL-1; IL-6; IL-8; IL-17; IL-21; IL-22; IL-27; IFN-gamma; neopterin; kynurenine; homocysteine; C-reactive protein; circulating complement degradation compounds; fibrinogen; D-Dimers; and HMGB-1

In one aspect of the invention a property associated with inflammation is selected from the group consisting of: erythrocyte sedimentation; monocyte production of TNF-alpha; monocyte production of TNF-alpha after LPS stimulation; expression of TLR-2 on monocytes; and expression of TLR-4 on monocytes.

### DETAILED DESCRIPTION

Embodiments of the present invention are described below. It is, however, expressly noted that the present invention is not limited to these embodiments, but rather the intention is that modifications that are apparent to the person skilled in the art and equivalents thereof are also included.

The endothelium is developed embryologically from the dual hematopoietic and endothelial progenitor, the hemangioblast [1]. It is believed that circulating endothelial progenitor cells (EPC) are in some ways related to this cell, being an “adult version” of it. Four decades ago a study supported the possibility of this “adult hemangioblast,” whose role is to replenish old/injured endothelium. Specifically, it was found that endothelial-like cells, that are non-thrombogenic and morphologically similar to endothelium are found on a plastic graft that was tethered to the thoracic artery of a pig [2].

More recent studies have performed molecular characterization of EPC. Specifically, a 1997 paper by Asahara et al. described EPC as bone marrow derived VEGR-2 positive, CD34 positive monocyte-like cells, having ability to differentiate into endothelial cells in vitro and in vivo based on expression of CD31, eNOS, and E-selectin [3]. One of the first examinations of circulating EPC was performed in the hindlimb ischemia model in mice and rabbit models. It was shown that there was an increase in circulation of EPC in response to ischemic insult [4]. Furthermore, these studies demonstrated that cytokine-induced augmentation of EPC mobilization elicited a therapeutic angiogenic response. Using irradiated chimeric systems, it was demonstrated that the ischemia-mobilized EPC derive from the bone marrow, and that these cells participate both in sprouting of pre-existing blood vessels as well as the initiation of de novo blood vessel production [5].

Subsequent to the initial phenotypic characterization by Asahara et al [3], more detailed descriptions of the human EPC were reported. For example, CD34 cells expressing the markers, VEGF-receptor 2, CD133, and CXCR-4 receptor, with migrational ability to VEGF and SDF-1 has been a more refined EPC definition [6]. However there is still some controversy as to the precise phenotype of the EPC, since the term implies only ability to differentiate into endothelium. Specifically, both CD34+, VEGFR2+, CD133+, as well as CD34+, VEGFR2+, CD133− have been reported to act as EPC [7]. More recent studies suggest that the subpopulation lacking CD133 and CD45 are phenotypically immature, precursor EPC [8]. Other phenotypes have been ascribed to cells with EPC activity, one study demonstrated monocyte-like cells that express CD14, Mac-1 and the dendritic cell marker CD11c have EPC activity based on uptake of acetylated LDL and binding to the ulex-lectin [9, 10].

Evidence supporting a role for EPC in vascular endothelial turnover came from studies in the apolipoprotein E knockout (ApoE KO) mouse, which are genetically predisposed to development of atherosclerosis due to inability to impair catabolism of triglyceride-rich lipoproteins. When these mice are lethally irradiated and reconstituted with labeled bone marrow stem cells, it was found that areas of the vasculature with high endothelial turnover, which were the areas of elevated levels of sheer stress, had incorporated the majority of new endothelial cells derived from the bone marrow EPC [11].

The possibility that endogenous bone marrow derived EPC possess such a regenerative function was also tested in a therapeutic setting. Atherosclerosis is believed to initiate from endothelial injury with a proliferative neointimal response that leads to formation of plaques. When bone marrow derived EPC are administered subsequent to wire injury, a substantial reduction in neointima formation was observed [12]. The argument can obviously made that wire injury of an artery does not resemble the physiological conditions associated with plaque development. To address this, Wassmann et al [13], used ApoE KO mice that were fed a high cholesterol diet and observed reduction in endothelial function as assessed by the flow mediated dilation assay. When EPC were administered from wild-type mice restoration of endothelial responsiveness was observed.

EPC have also been demonstrated to possess an anti-aging function in a series of experiments in which 3 month old syngeneic cardiac grafts were heterotopically implanted into 18 month old recipients. Loss of graft viability, associated with poor neovascularization, was observed subsequent to transplanting, as well as subsequent to administration of 18 month old bone marrow mononuclear cells. In contrast, when 3 month old bone marrow mononuclear cells were implanted, grafts survived. Antibody depletion experiments demonstrated bone marrow derived PDGF-BB was essential in integration of the young heart cells with the old recipient vasculature [14]. These experiments suggest that young EPC or EPC-like cells have ability to integrate and interact with older vasculature.

In one aspect of the present technology, EPC are quantified as a general measurement of health, and specifically, to assess the response to naturopathic intervention. For example, the food supplement citicholine is used by many naturopaths to “enhance thinking clarity.” Citicoline is a chemical that is found naturally in the body that acts as an intermediate in the generation of phosphatidylcholine from choline. It is sold as a food supplement in the USA. A recent study (Sobrino et al. CDP-choline treatment increases circulating endothelial progenitor cells in acute ischemic stroke. Neurol Res. 2011 July; 33(6):572-7) demonstrated that this food supplement appears to increase numbers of circulating EPC. Forty eight stroke patients were randomized into treatment (26 patients) and placebo (22 patients). The treatment consisted of daily administration of 2000 mg of citicoline per day for 6 weeks after the stroke. A statistically significant increase in EPC was noted at day 7 after treatment with citicoline. Patients who received clotbuster drugs together with citicoline also had higher EPC numbers. Given that citicoline appears to have some therapeutic effects in stroke, traumatic brain injury and cognitive impairment (as reviewed in Secades J J, Citicoline: pharmacological and clinical review, 2010 update. Rev Neurol 2011 Mar. 14; 52 Suppl 2:S1-S62), it may be possible that some of the effects mediated by this food supplement are associated with its ability to increase circulating EPC. One embodiment of the current present technology is to provide EPC numbers in patients taking supplements such as citicoline, correlate its EPC augmenting effect in specific patients, and determine whether other interventions are needed to obtain an “optimized” EPC number in circulation. In some embodiments, an optimized EPC number can be in reference to levels determined across a general population of patients, or within a specific subpopulation. In some embodiments, optimized EPC numbers can vary for an individual patient. For example, where a patent has been previously tested for circulating EPC levels, the optimized EPC number for that patient can be a threshold known to correlate with improved or improving health for that specific patient. This optimized number may also be correlated with other parameters within the scope of the present technology. For example, inflammatory markers may be assessed in combination.

Detection of circulating EPC can be important in response to therapies such as hyperbaric oxygen, which is commonly used by naturopathic physicians. Thom et al [15] examined diabetic patients who underwent therapy for hyperbaric oxygen. They reported more than a twofold elevation (p=0.004) in circulating stem cells after treatments. Interestingly the circulating stem cells, which were quantified based on expression of CD34, contained two- to threefold higher levels of hypoxia inducible factors-1, -2, and -3, as well as thioredoxin-1 (p<0.003), than cells present in blood before hyperbaric oxygen treatment or therapy (HBO(2)T). They further found that nitric oxide synthase activity is acutely increased in patients' platelets following HBO(2)T and remains elevated for at least 20 hours. Mechanistically they proposed hyperbaric oxygen increases circulating stem cells through stimulation of nitric oxide production [16]. Without being bound to theory, it appears that the intervention of hyperbaric oxygen treatment increases stem cells in circulation [17-20], and others have found that increased stem cells in circulation correlate with better prognosis for a variety of indications. However, to our knowledge, the personalized use of circulating regenerative cell testing as a guide for administration of naturopathic interventions has not been practiced.

Numerous strategies that are used in the context of naturopathic medicine seem to mediate their activities through the inhibition of inflammation. Previously it has been shown that inhibitors of inflammation increase EPC numbers. Various agents are known to decrease inflammation, these include TNF blockers such as remicade [21], consumption of various dietary supplements [22, 23], caloric restriction [24], exercise [25, 26], eating blueberries [27], green tea [28], or statin therapy [29]. The drug Crestor has been shown to increase circulating EPC levels in vivo [30], in part through reduction of detrimental effects of asymmetric dimethylarginine on EPC [31].

Granulocyte colony stimulating factor (G-CSF) has been used clinically for mobilization of hematopoietic stem cells (HSC) for more than a decade during donor stem cell harvesting. Mechanistically G-CSF is believed to induce a MMP-dependent alteration of the SDF-1 gradient in the bone marrow [32, 33], as well as function through a complement-dependent remodeling of the bone marrow extracellular matrix [34, 35]. It was found that in addition to mobilizing HSC, G-CSF stimulates mobilization of EPC as well, through mechanisms that are believed to be related [7, 36]. Several studies have been performed in which G-CSF was administered subsequent to infarct. Although it is impossible to state whether the mobilization of HSC or EPC accounted for the beneficial effects, we will overview some of these studies. The Front-Integrated Revascularization and Stem Cell Liberation in Evolving Acute Myocardial Infarction by Granulocyte Colony-Stimulating Factor (FIRSTLINE-AMI) trial evaluated 30 patients with ST-elevation myocardial infarction treated with control or G-CSF after successful revascularization [37]. Fifteen patients received 6 days of G-CSF at 10 μg/kg body weight, whereas the other 15 received standard care only. Four months after the infarct, the group that received G-CSF possessed a thicker myocardial wall at the area of infarct, as compared to controls. This was sustained over a year. Statistically significant improvements in ejection fraction, as well as inhibition of pathological remodeling was observed in comparison to controls. A larger subsequent study with 114 patients, 56 treated and 58 control demonstrated “no influence on infarct size, left ventricular function, or coronary restenosis” [38]. There may be a variety of reasons to explain the discrepancy between the trials. One most obvious one is that the mobilization was conducted immediately after the heart attack, whereas it may be more beneficial to time the mobilization with the timing of the chemotactic gradient released by the injured myocardium. This has been used to explain discrepancies between similar regenerative medicine trials [39]. Supporting this possibility is a study in which altered dosing was used for the successful improvement in angina [40]. Furthermore, a recent study last year demonstrated that in 41 patients with large anterior wall AMI an improvement in lef ventricle ejection fraction (LVEF) and diminished pathological remodeling was observed [41]. There is an indication that post-infarct mobilization can have a therapeutic role. Other clinically-applicable mobilizers may be evaluated and used in the methods described herein. For example, growth hormone, which is used in “antiaging medicine” has been demonstrated to improve endothelial responsiveness in healthy volunteers [42], and patients with congestive heart failure [43], this appears to be mediated through mobilization of endothelial progenitor cells [44, 45]. Thus in one embodiment of the present technology, the “optimizing” of EPC levels for a particular health situation is performed by being able to monitor EPC levels and utilizing various interventions as needed. For example, interventions can include continuing with a course of treatment, discontinuing a course of treatment, increasing or decreasing the frequency or dosage levels of one or more treatments. In certain embodiments, the intervention can be a naturopathic intervention. In some embodiments, the naturopathic interventions is selected from the group consisting of: recommendations of lifestyle modification, dietary supplements, intravenous vitamins, detoxification, acupuncture, and guided imagery.

Having generally described this technology, a further understanding can be obtained by reference to certain specific examples which are provided herein for purposes of illustration only, and are not intended to be limiting.

### EXAMPLES

Within the context of the present technology, other cells besides EPC may be used as circulating regenerative cells. In one embodiment of the present technology, quantification of circulating very small embryonic like cells (VSEL) is performed as a marker of general health. Specifically, EDTA-anticoagulated peripheral samples (2×2.7 mL) are drawn from patients. The absolute numbers of leukocytes and lymphocytes in said peripheral blood are determined at the same time with an automatic cell counter, for example, using the Cell-Dyn 3500, Abbott Diagnostics, Santa Clara, Calif.). In order to purify peripheral blood mononuclear cells (PBMC), lysis of red blood cells is performed using a hypotonic lysis buffer, for example, the BD Pharm Lyse Buffer (BD Biosciences Pharmingen, San Diego, Calif.). The PBMC are subsequently used for flow cytometric analysis to determine VSEL content. Typically, care can be taken to reproducibly utilize the same reagent batches for consistency of testing. The expression of CD34, CD133, and CXCR4 on human PBMNCs is evaluated by flow cytometry. The staining and analyses of protein marker expression are performed by staining in phosphate-buffered saline (PBS; Ca2+- and Mg2+-free) supplemented with 5% bovine calf serum (Hyclone, Logan, Utah).

The following monoclonal antibodies directly conjugated with phycoerythrin (PE) or allophycocyanin (APC) may be used for quantification: PE-anti-CD34 (BD Biosciences Pharmingen); APC-anti-CXCR4 (BD Biosciences Pharmingen); and APC-anti-CD133 (Miltenyi Biotec, Auburn, Calif.). To determine the proportion of CD34+/CD133+, CXCR4+/CD34+, and CXCR4−/CD34+ cells, a dual-color flow cytometric analysis is performed. However, for this to occur, it can be helpful to utilize an appropriate isotype control (BD Biosciences Pharmingen). For staining, PBMNCs 10⁶ are mixed with antibody stained, and after a 20-minute incubation on ice, cells were washed twice in PBS. Thereafter, the cell pellet is resuspended in 0.3 mL PBS and analyzed by FACSAria (BD Biosciences, San Jose, Calif.) and Cell Quest software (BD Biosciences). Typically, 50,000 events are acquired to determine the percentage of the examined subpopulation within the PBMC population.

In other experiments, a single-cell suspension is stained for lineage markers (CD56, CD235a, CD3, CD66b, CD24, CD19, CD14, CD16, CD2) conjugated with fluorescein isothiocyanate, CD45 conjugated with PE, and CXCR4 conjugated with APC for 30 minutes on ice. After being washed, cells are analyzed by fluorescence-activated cell sorting (BD Biosciences). At least 10⁵ events were acquired and analyzed by Cell Quest software (BD Biosciences). In one embodiment, VSEL are quantified as lin− CD45− CXCR4+ cells of a small (less than 7 microns) size. While others have demonstrated that VSEL cells increase in circulation after heart attack [46], and stroke [47], the surprising discovery of using these cells as a general marker of health represents a key advancement in the field.

The above description discloses several methods and systems of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein including, but not limited to, published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

The following references have been referred to in the text. The entire content of each reference is incorporated herein. Without limitation all of the cells, proteins, markers, assays, and methods can be used with the technology described herein and/or can be combined with the methods and materials described herein.

### REFERENCES

1. Basak, G. W., et al., Human embryonic stem cells hemangioblast express HLA-antigens. J Transl Med, 2009. 7: p. 27.
2. Stump, M. M., et al., Endothelium Grown from Circulating Blood on Isolated Intravascular Dacron Hub. Am J Pathol, 1963. 43: p. 361-7.
3. Asahara, T., et al., Isolation of putative progenitor endothelial cells for angiogenesis. Science, 1997. 275(5302): p. 964-7.
4. Takahashi, T., et al., Ischemia- and cytokine-induced mobilization of bone marrow-derived endothelial progenitor cells for neovascularization. Nat Med, 1999. 5(4): p. 434-8.
5. Asahara, T., et al., Bone marrow origin of endothelial progenitor cells responsible for postnatal vasculogenesis in physiological and pathological neovascularization. Circ Res, 1999. 85(3): p. 221-8.
6. Peichev, M., et al., Expression of VEGFR-2 and AC133 by circulating human CD34(+) cells identifies a population of functional endothelial precursors. Blood, 2000. 95(3): p. 952-8.
7. Korbling, M., et al., Recombinant human granulocyte-colony-stimulating factor-mobilized and apheresis-collected endothelial progenitor cells: a novel blood cell component for therapeutic vasculogenesis. Transfusion, 2006. 46(10): p. 1795-802.
8. Timmermans, F., et al., Endothelial outgrowth cells are not derived from CD133+ cells or CD45+ hematopoietic precursors. Arterioscler Thromb Vasc Biol, 2007. 27(7): p. 1572-9.
9. Rehman, J., et al., Peripheral blood “endothelial progenitor cells” are derived from monocyte/macrophages and secrete angiogenic growth factors. Circulation, 2003. 107(8): p. 1164-9.
10. Rohde, E., et al., Blood monocytes mimic endothelial progenitor cells. Stem Cells, 2006. 24(2): p. 357-67.
11. Foteinos, G., et al., Rapid endothelial turnover in atherosclerosis prone areas coincides with stem cell repair in apolipoprotein E-deficient mice. Circulation, 2008. 117(14): p. 1856-63.
12. Werner, N., et al., Intravenous transfusion of endothelial progenitor cells reduces neointima formation after vascular injury. Circ Res, 2003. 93(2): p. e17-24.
13. Wassmann, S., et al., Improvement of endothelial function by systemic transfusion of vascular progenitor cells. Circ Res, 2006. 99(8): p. e74-83.
14. Edelberg, J. M., et al., Young adult bone marrow-derived endothelial precursor cells restore aging-impaired cardiac angiogenic function. Circ Res, 2002. 90(10): p. E89-93.
15. Thom, S. R., et al., Vasculogenic stem cell mobilization and wound recruitment in diabetic patients: increased cell number and intracellular regulatory protein content associated with hyperbaric oxygen therapy. Wound repair and regeneration: official publication of the Wound Healing Society [and] the European Tissue Repair Society, 2011. 19(2): p. 149-61.
16. Goldstein, L. J., et al., Endothelial progenitor cell release into circulation is triggered by hyperoxia-induced increases in bone marrow nitric oxide. Stem cells, 2006. 24(10): p. 2309-18.
17. Liu, Z. J. and O. C. Velazquez, Hyperoxia, endothelial progenitor cell mobilization, and diabetic wound healing. Antioxidants & redox signaling, 2008. 10(11): p. 1869-82.
18. Wang, X. L., et al., Therapeutic window of hyperbaric oxygen therapy for hypoxic-ischemic brain damage in newborn rats. Brain research, 2008. 1222: p. 87-94.
19. Velazquez, O. C., Angiogenesis and vasculogenesis: inducing the growth of new blood vessels and wound healing by stimulation of bone marrow-derived progenitor cell mobilization and homing. Journal of vascular surgery: official publication, the Society for Vascular Surgery [and] International Society for Cardiovascular Surgery, North American Chapter, 2007. 45 Suppl A: p. A39-47.
20. Thom, S. R., et al., Stem cell mobilization by hyperbaric oxygen. American journal of physiology. Heart and circulatory physiology, 2006. 290(4): p. H1378-86.
21. Wu, J., et al., Intravenously administered bone marrow cells migrate to damaged brain tissue and improve neural function in ischemic rats. Cell Transplant, 2008. 16(10): p. 993-1005.
22. Phillips, T., et al., A dietary supplement attenuates IL-6 and CRP after eccentric exercise in untrained males. Med Sci Sports Exerc, 2003. 35(12): p. 2032-7.
23. Regensteiner, J. G., et al., Oral L-arginine and vitamins E and C improve endothelial function in women with type 2 diabetes. Vasc Med, 2003. 8(3): p. 169-75.
24. Kalani, R., et al., Effects of caloric restriction and exercise on age-related, chronic inflammation assessed by C-reactive protein and interleukin-6. J Gerontol A Biol Sci Med Sci, 2006. 61(3): p. 211-7.
25. Colbert, L. H., et al., Physical activity, exercise, and inflammatory markers in older adults: findings from the Health, Aging and Body Composition Study. J Am Geriatr Soc, 2004. 52(7): p. 1098-104.
26. Thijssen, D. H., et al., Vascular adaptations to 8-week cycling training in older men. Acta Physiol (Oxf), 2007. 190(3): p. 221-8.
27. Abidov, M., et al., Effect of Blueberin on fasting glucose, C-reactive protein and plasma aminotransferases, in female volunteers with diabetes type 2: double-blind, placebo controlled clinical study. Georgian Med News, 2006(141): p. 66-72.
28. Alexopoulos, N., et al., The acute effect of green tea consumption on endothelial function in healthy individuals. Eur J Cardiovasc Prev Rehabil, 2008. 15(3): p. 300-5.
29. Ridker, P. M., et al., Rosuvastatin to prevent vascular events in men and women with elevated C-reactive protein. N Engl J Med, 2008. 359(21): p. 2195-207.
30. Spiel, A. O., et al., Simvastatin and rosuvastatin mobilize Endothelial Progenitor Cells but do not prevent their acute decrease during systemic inflammation. Thromb Res, 2008. 123(1): p. 108-13.
31. Thum, T., et al., Suppression of endothelial progenitor cells in human coronary artery disease by the endogenous nitric oxide synthase inhibitor asymmetric dimethylarginine. J Am Coll Cardiol, 2005. 46(9): p. 1693-701.
32. Jin, F., et al., Degradation of BM SDF-1 by MMP-9: the role in G-CSF-induced hematopoietic stem/progenitor cell mobilization. Bone Marrow Transplant, 2008. 42(9): p. 581-8.
33. Carion, A., et al., Stromal-derived factor 1 and matrix metalloproteinase 9 levels in bone marrow and peripheral blood of patients mobilized by granulocyte colony-stimulating factor and chemotherapy. Relationship with mobilizing capacity of haematopoietic progenitor cells. Br J Haematol, 2003. 122(6): p. 918-26.
34. Ratajczak, M. Z., et al., A pivotal role of activation of complement cascade (CC) in mobilization of hematopoietic stem/progenitor cells (HSPC). Adv Exp Med Biol, 2008. 632: p. 47-60.
35. Lee, H. M., et al., Impaired mobilization of hematopoietic stem/progenitor cells in CS-deficient mice supports the pivotal involvement of innate immunity in this process and reveals novel promobilization effects of granulocytes. Leukemia, 2009.
36. Pitchford, S. C., et al., Differential mobilization of subsets of progenitor cells from the bone marrow. Cell Stem Cell, 2009. 4(1): p. 62-72.
37. Ince, H., et al., Prevention of left ventricular remodeling with granulocyte colony-stimulating factor after acute myocardial infarction: final 1-year results of the Front-Integrated Revascularization and Stem Cell Liberation in Evolving Acute Myocardial Infarction by Granulocyte Colony-Stimulating Factor (FIRSTLINE-AMI) Trial. Circulation, 2005. 112(9 Suppl): p. 173-80.
38. Zohlnhofer, D., et al., Stem cell mobilization by granulocyte colony-stimulating factor in patients with acute myocardial infarction: a randomized controlled trial. JAMA, 2006. 295(9): p. 1003-10.
39. Hill, J. M. and J. Bartunek, The end of granulocyte colony-stimulating factor in acute myocardial infarction? Reaping the benefits beyond cytokine mobilization. Circulation, 2006. 113(16): p. 1926-8.
40. Suzuki, K., et al., Effect of granulocyte colony-stimulating factor treatment at a low dose but for a long duration in patients with coronary heart disease. Circ J, 2006. 70(4): p. 430-7.
41. Leone, A. M., et al., Usefulness of granulocyte colony-stimulating factor in patients with a large anterior wall acute myocardial infarction to prevent left ventricular remodeling (the rigenera study). Am J Cardiol, 2007. 100(3): p. 397-403.
42. Napoli, R., et al., Acute effects of growth hormone on vascular function in human subjects. J Clin Endocrinol Metab, 2003. 88(6): p. 2817-20.
43. Napoli, R., et al., Growth hormone corrects vascular dysfunction in patients with chronic heart failure. J Am Coll Cardiol, 2002. 39(1): p. 90-5.
44. Devin, J. K., et al., Low-dose growth hormone administration mobilizes endothelial progenitor cells in healthy adults. Growth Horm IGF Res, 2008. 18(3): p. 253-63.
45. Thum, T., et al., Age-dependent impairment of endothelial progenitor cells is corrected by growth-hormone-mediated increase of insulin-like growth-factor-1. Circ Res, 2007. 100(3): p. 434-43.
46. Wojakowski, W., et al., Mobilization of bone marrow-derived Oct-4+ SSEA-4+ very small embryonic-like stem cells in patients with acute myocardial infarction. Journal of the American College of Cardiology, 2009. 53(1): p. 1-9.
47. Paczkowska, E., et al., Clinical evidence that very small embryonic-like stem cells are mobilized into peripheral blood in patients after stroke. Stroke; a journal of cerebral circulation, 2009. 40(4): p. 1237-44.

1. A method of assessing the general health of a mammal, the method comprising:
a) obtaining a blood sample;
b) labeling said blood sample with an agent capable of selectively binding a cell with regenerative potential;
c) quantifying a number of cells with regenerative potential; and
d) relating said number of cells with regenerative potential with various aspects of general health of said mammal.

2. The method of claim 1, further comprising:
e) prescribing a naturopathic intervention based upon the quantifying or relating.

3. The method of claim 1, further comprising:
e) performing or providing a naturopathic intervention based upon the quantifying or relating.

4. The method of any of claims 2-3, wherein said naturopathic intervention is selected from the group consisting of: recommendations of lifestyle modification, dietary supplements, intravenous vitamins, detoxification, acupuncture, and guided imagery.

5. The method of claim 1, wherein said agent capable of selectively binding a cell with regenerative potential is selected from the group consisting of: a protein; a peptide; a nucleic acid; and a small molecule.

6. The method of claim 5, wherein said agent capable of selectively binding a cell with regenerative potential is an antibody.

7. The method of claim 5 or claim 6, wherein said agent capable of selectively binding a cell with regenerative potential is further conjugated to a label enabling detection.

8. The method of claim 1, wherein said cell with regenerative potential is a circulating cell selected from the group consisting of mesenchymal stem cells (MSC); endothelial progenitor cells (EPC); very small embryonic like cells (VSEL); and hematopoietic stem cells.

9. The method of claim 1, wherein said cell with regenerative potential is a circulating endothelial progenitor cell.

10. The method of claim 8, wherein said mesenchymal stem cells are detected by an agent capable of binding a molecule selected from the group consisting of: CD5; CD9; CD73; CD90; and CD105.

11. The method of claim 10, wherein said mesenchymal stem cells are detected by absence of expression of a marker selected from the group consisting of: CD14; CD34; CD45; and HLA II.

12. The method of claim 8, wherein said endothelial progenitor cells are detected by an agent capable of binding a molecule selected from the group consisting of: CD34; CD133; KDR-1; and CD166.

13. The method of claim 8, wherein said very small embryonic like cells are detected based on a size of less than 7 microns together with expression of a molecule selected from the group consisting of: wnt-5; CD34; CD133; Oct-4; Nanog; and SSEA-1.

14. The method of claim 8, wherein said hematopoietic stem cells are detected based on expression of a molecule selected from the group consisting of: CD34; CD133; c-kit; and jagged.

15. The method of claim 14, wherein said hematopoietic stem cells express less than 5% of markers selected from the group consisting of: CD14; CD38; and CD45.

16. The method of claim 8, wherein said mesenchymal stem cell is capable of forming mesenchymal stem cells when cultured in liquid culture, said mesenchymal stem cells having ability to differentiate into osteocytic, chondrocytic, and adipocytic lineages.

17. The method of claim 8, wherein said endothelial progenitor cell is capable of forming endothelial cells when cultured in liquid culture, said endothelial cells expressing ability to uptake acetylated LDL.

18. The method of claim 8, wherein said very small embryonic like cell is capable of forming multiple tissues in liquid culture in addition to adipose, chondrogenic, and osteogenic tissues.

19. The method of claim 8, wherein said hematopoietic stem cell is capable of forming hematopoietic tissues in liquid culture, said hematopoietic tissues including granulocytes and monocytes.

20. The method of any of claim 8, wherein the quantifying of circulating regenerative cells is performed by flow cytometry.
